(19) United States
(12) Patent Application Publication
(10) Pub. No.: US 2016/0157832 A1
(43) Pub. Date: Jun. 9, 2016

(54) APPARATUS AND METHOD FOR COMPUTER AIDED DIAGNOSIS (CAD), AND APPARATUS FOR CONTROLLING ULTRASONIC TRANSMISSION PATTERN OF PROBE

(71) Applicant: SAMSUNG ELECTRONICS CO., LTD., Suwon-si (KR)

(72) Inventors: Byung Kon KANG, Gwangju-si (KR); Jung Hoe KIM, Seongnam-si (KR); Hyoung Min PARK, Seoul (KR)

(73) Assignee: SAMSUNG ELECTRONICS CO., LTD., Suwon-si (KR)

(21) Appl. No.: 14/958,144

(22) Filed: Dec. 3, 2015

(30) Foreign Application Priority Data
Dec. 3, 2014 (KR) 10-2014-0172349

(51) Int. Cl. A61B 8/00; G06T 7/00; G06K 9/32; A61B 8/08; G06K 9/62

(52) U.S. Cl. CPC A61B 8/54; A61B 8/5207; A61B 8/5215; G06K 9/6284; G06K 9/3233; G06T 7/0016; G06T 2207/10132

(57) ABSTRACT

An apparatus and a method for Computer Aided Diagnosis (CAD) and an apparatus for controlling an ultrasonic transmission pattern of a probe, are provided. The apparatus for controlling the ultrasonic transmission pattern is configured to transmit ultrasonic signals to an object in directions, and receive ultrasonic echo signals in response to the ultrasonic signals being reflected from the object. The apparatus for controlling the ultrasonic transmission pattern includes a determiner configured to determine, based on pressure that is applied on the probe, a number of transmission directions in which the ultrasonic signals are to be transmitted, and determine the transmission directions in which the ultrasonic signals are to be transmitted, based on the determined number of the transmission directions. The apparatus further includes an energy controller configured to control energy of each of the ultrasonic signals based on the determined number of the transmission directions.

### CROSS-REFERENCE TO RELATED APPLICATION

This application claims priority from Korean Patent Application No. 10-2014-0172349, filed on Dec. 3, 2014, in the Korean Intellectual Property Office, the disclosure of which is incorporated herein by reference in its entirety.

### BACKGROUND

1. Field

Apparatuses and methods consistent with exemplary embodiments relate to an apparatus and a method for Computer Aided Diagnosis (CAD), and an apparatus for controlling an ultrasonic transmission pattern of a probe.

2. Description of the Related Art

A Computer Aided Diagnosis (CAD) system is a system that analyzes medical images, i.e., ultrasonic images, and displays a suspicious area on a medical image according to a diagnostic result to help a doctor to diagnose a patient's disease. It is almost impossible for humans to perform error-free diagnosis because of their limited perceptive capability. In addition, analyzing each medical image involves great attention and care. The CAD system can help raise accuracy of diagnosis and alleviate burden on doctors.

A conventional CAD system transmits ultrasonic waves in a unilateral direction using a probe to acquire an ultrasonic image of a lesion. In this system, ultrasonic images may be changed according to an angle at which a probe is placed on a patient's skin, thereby affecting an automatic classification process. It may cause confusion when ultrasonic images of a tumor are captured, because similar ultrasonic images may be generated according to the insonation angle relative to the skin.

### SUMMARY

Exemplary embodiments address at least the above problems and/or disadvantages and other disadvantages not described above. Also, the exemplary embodiments are not required to overcome the disadvantages described above, and may not overcome any of the problems described above.

According to an aspect of an exemplary embodiment, there is provided an apparatus for controlling an ultrasonic transmission pattern of a probe configured to transmit ultrasonic signals to an object in directions, and receive ultrasonic echo signals in response to the ultrasonic signals being reflected from the object, the apparatus including a determiner configured to determine, based on pressure that is applied on the probe, a number of transmission directions in which the ultrasonic signals are to be transmitted, and determine the transmission directions in which the ultrasonic signals are to be transmitted, based on the determined number of the transmission directions. The apparatus further includes an energy controller configured to control energy of each of the ultrasonic signals based on the determined number of the transmission directions.

The determiner may be further configured to determine the number of the transmission directions to be inversely proportional to the pressure applied on the probe.

The determiner may be further configured to select the transmission directions from available directions in which the probe is capable of transmitting the ultrasonic signals, based on a closeness of an angle between each of the available directions and an axis vertical to a surface of the object in contact with the probe, and a number of the selected transmission directions may correspond to the determined number of the transmission directions.

The energy controller may be further configured to control the energy of each of the ultrasonic signals to be inversely proportional to the determined number of the transmission directions.

The pressure applied on the probe may be from a surface of the object in contact with the probe, or from a holder of the probe.

The apparatus may further include a pressure sensor configured to sense the pressure applied on the probe.

According to an aspect of another exemplary embodiment, there is provided a Computer Aided Diagnosis (CAD) apparatus using a probe configured to transmit ultrasonic signals to an object in directions, and receive ultrasonic echo signals in response to the ultrasonic signals being reflected from the object, the apparatus including an ultrasonic transmission pattern controller configured to determine, based on pressure that is applied on the probe, a number of transmission directions in which the ultrasonic signals are to be transmitted, and determine the transmission directions in which the ultrasonic signals are to be transmitted, based on the determined number of the transmission directions. The CAD apparatus further includes an image generator configured to generate ultrasonic images based on the ultrasonic echo signals that are received in response to the ultrasonic signals being transmitted to the object in the determined transmission directions, a number of the generated ultrasonic images corresponding to the determined number of the transmission directions. The CAD apparatus further includes a classifier configured to classify each of the generated ultrasonic images, and combine results of the classification to generate a final result of the classification.

The ultrasonic transmission pattern controller may be further configured to determine the number of the transmission directions to be inversely proportional to the pressure applied on the probe.

The ultrasonic transmission pattern controller may be further configured to select the transmission directions from available directions in which the probe is capable of transmitting the ultrasonic signals, based on a closeness of an angle between each of the available directions and an axis vertical to a surface of the object in contact with the probe, and a number of the selected transmission directions may correspond to the determined number of the transmission directions.

The ultrasonic transmission pattern controller may be further configured to control energy of each of the ultrasonic signals based on the determined number of the transmission directions.

The ultrasonic transmission pattern controller may be further configured to control the energy of each of the ultrasonic signals to be inversely proportional to the determined number of the transmission directions.

The pressure applied on the probe may be from a surface of the object in contact with the probe, or from a holder of the probe.

The CAD apparatus may further include a region of interest (ROI) detector configured to detect an ROI from each of the generated ultrasonic images, and the classifier may be further configured to exclude, from the classification, an ultrasonic image in which an ROI is not detected among the generated ultrasonic images.

According to an aspect of another exemplary embodiment, there is provided a Computer Aided Diagnosis (CAD) method using a probe configured to transmit ultrasonic signals to an object in directions, and receive ultrasonic echo signals in response to the ultrasonic signals being reflected from the object, the method including determining, based on pressure that is applied on the probe, a number of transmission directions in which the ultrasonic signals are to be transmitted, and determining the transmission directions in which the ultrasonic signals are to be transmitted, based on the determined number of the transmission directions. The CAD method further includes generating ultrasonic images based on the ultrasonic echo signals that are received in response to the ultrasonic signals being transmitted to the object in the determined transmission directions, a number of the generated ultrasonic images corresponding to the determined number of the transmission directions. The CAD method further includes classifying each of the generated ultrasonic images, and combining results of the classification to generate a final result of the classification.

The determining the number of the transmission directions may include determining the number of the transmission directions to be inversely proportional to the pressure applied on the probe.

The determining the transmission directions may include selecting the transmission directions from available directions in which the probe is capable of transmitting the ultrasonic signals, based on a closeness of an angle between each of the available directions and an axis vertical to a surface of the object in contact with the probe, and a number of the selected transmission directions may correspond to the determined number of the transmission directions.

The CAD method may further include controlling energy of each of the ultrasonic signals based on the determined number of the transmission directions.

The controlling of energy may include controlling the energy of each of the ultrasonic signals to be inversely proportional to the determined number of the transmission directions.

The pressure applied on the probe may be from a surface of the object in contact with the probe, or from a holder of the probe.

The CAD method may further include detecting a region of interest (ROI) from each of the generated ultrasonic images, and the classifying may include excluding, from the classification, an ultrasonic area in which an ROI is not detected among the generated ultrasonic images.

### BRIEF DESCRIPTION OF THE DRAWINGS

The above and/or other aspects will be more apparent by describing exemplary embodiments, with reference to the accompanying drawings, in which:

FIG. 1 is a block diagram illustrating a Computer Aided Diagnosis (CAD) system, according to an exemplary embodiment;

FIG. 2 is a block diagram illustrating a CAD apparatus shown in FIG. 1;

FIG. 3 is a block diagram illustrating an ultrasonic transmission pattern controller shown in FIG. 2;

FIG. 4 is a diagram illustrating a number of transmission directions being changed according to a change in pressure on a probe, according to an exemplary embodiment;

FIG. 5 is a diagram illustrating operations of the CAD apparatus shown in FIG. 2;

FIG. 6 is a flowchart illustrating a CAD method, according to an exemplary embodiment; and

FIG. 7 is a flowchart illustrating an operation of determining a number of transmission directions in which ultrasonic signals are to be transmitted and the transmission directions in which ultrasonic signals are to be transmitted, which is shown in FIG. 6.

### DETAILED DESCRIPTION

Exemplary embodiments are described in greater detail below with reference to the accompanying drawings.

In the following description, like drawing reference numerals are used for like elements, even in different drawings. The matters defined in the description, such as detailed construction and elements, are provided to assist in a comprehensive understanding of the exemplary embodiments. However, it is apparent that the exemplary embodiments can be practiced without those specifically defined matters. Also, well-known functions or constructions may not be described in detail because they would obscure the description with unnecessary detail.

It will be understood that the terms “comprises” and/or “comprising” used herein specify the presence of stated features or components, but do not preclude the presence or addition of one or more other features or components. In addition, the terms such as “unit”, “-er (-or)”, and “module” described in the specification refer to an element for performing at least one function or operation, and may be implemented in hardware, software, or the combination of hardware and software.

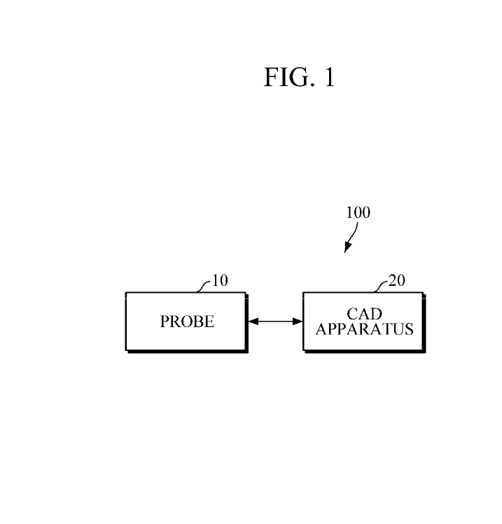

FIG. 1 is a block diagram illustrating a Computer Aided Diagnosis (CAD) system 100, according to an exemplary embodiment.

Referring to FIG. 1, the CAD system 100 includes a probe 10 and a CAD apparatus 20.

The probe 10 may transmit ultrasonic signals to an object in multiple directions simultaneously or sequentially, and receive an ultrasonic echo signal that is an ultrasonic signal reflected from the object. The CAD apparatus 20 determines: the number of transmission directions in which ultrasonic signals are to be transmitted; the transmission directions in which the ultrasonic signals are to be transmitted; and energy of each ultrasonic signal. Then, the probe 10 may transmit the ultrasonic signals with the determined energy to the object in the determined transmission directions.

According to pressure applied on the probe 10, the CAD apparatus 20 may determine the number of transmission directions in which ultrasonic signals are to be transmitted, the transmission directions in which ultrasonic signals are to be transmitted, and energy of each ultrasonic signal to be transmitted from the probe 10.

In addition, the CAD apparatus 20 may generate ultrasound images of an object based on ultrasonic echo signals that are received in response to ultrasonic signals transmitted to the object in multiple directions. The number of generated ultrasound images may be the same as the number of transmission directions in which the ultrasonic signals have been transmitted by the probe 10.

The CAD apparatus 20 may perform multi-view classification on the generated ultrasound images. The multi-view classification refers to a technique of performing a classification on an object by combining multiple images that show different characteristics of the object.

That is, the CAD apparatus 20 may control the number of ultrasonic images to be acquired, by controlling the number of transmission directions in which the ultrasonic signals are to be transmitted according to pressure applied on the probe 10, and classify a lesion by combining the acquired ultrasonic images. In addition, if greater power is applied on the probe 10, the CAD apparatus 20 may reduce more transmission directions, in which ultrasonic signals are to be transmitted, and increase energy of each ultrasonic signal, thereby obtaining a fewer number of high-definition ultrasonic images.

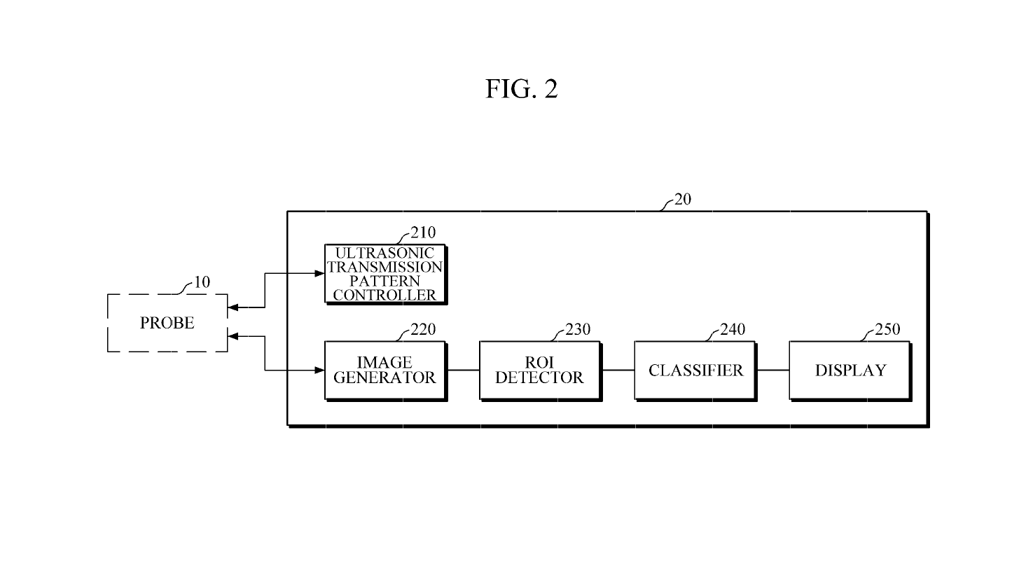

Hereinafter, the CAD apparatus 20 is described in detail with reference to FIG. 2.

FIG. 2 is a block diagram illustrating the CAD apparatus 20 shown in FIG. 1.

Referring to FIG. 2, the CAD apparatus 20 includes an ultrasonic transmission pattern controller 210, an image generator 220, a region of interest (ROI) detector 230, a classifier 240, and a display 250.

According to pressure applied on the probe 10, the ultrasonic transmission pattern controller 210 may determine the number of transmission directions in which ultrasonic signals are to be transmitted and the transmission directions in which ultrasonic signals are to be transmitted. In addition, according to the number of the transmission directions in which ultrasonic signals are to be transmitted, the ultrasonic transmission pattern controller 210 may control energy of each ultrasonic signal to be transmitted by the probe 10.

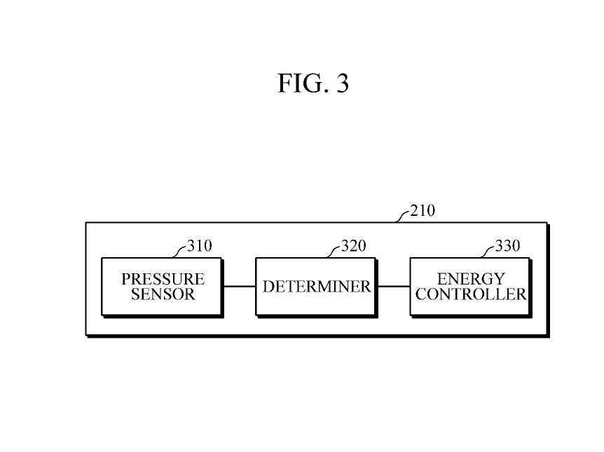

The ultrasonic transmission pattern controller 210 is described in detail with reference to FIG. 3.

The image generator 220 may generate an ultrasonic image of an object based on ultrasonic echo signals that are received in response to ultrasonic signals transmitted from the determined directions. The number of ultrasonic images to be generated may be the same as the determined number of transmission directions in which ultrasonic signals are to be transmitted. For example, when ultrasonic echo signals are received in three directions as the probe 10 transmits ultrasonic signals in the three directions simultaneously or sequentially, the CAD apparatus 20 may generate three ultrasonic images for the three transmission directions, respectively.

The classifier 240 may perform multi view classification on the generated ultrasonic images. According to an exemplary embodiment, the classifier 240 may extract features from each generated ultrasonic image, compare the extracted features with a pre-stored diagnostic model to perform a classification on each ultrasonic image, and compute a conclusive classification result by combining the classification results (e.g., benignancy/malignancy and confidence on determining as benignancy/malignancy).

A feature may be a factor that is considered to determine whether there is a lesion or not. For example, a feature may be a feature of a lesion (e.g., shape, margin, echo pattern, orientation, boundary, and the like) according to Breast Imaging Reporting and Data System (BI-RADS) Lexicon classification.

The diagnostic model may be generated by performing machine learning using features extracted from a plurality of pre-collected diagnostic images, and, once generated, may be stored in the classifier 240 or an external database.

A machine learning algorithm may include artificial neural network, decision tree, Genetic Algorithm (GA), Genetic Programming (GP), Gauss-Jordan Elimination, linear regression analysis, K-Nearest Neighbor (K-NN), perceptron, Radial Basis Function Networks (RBFN), Support Vector Machine (SVM), and the like.

The ROI detector 230 may detect an ROI from each ultrasonic image using a lesion detection algorithm. The ROI may include not only a malignant lesion area, but also a lesion area that is not definitely determined to be malignant/benign or has unusual features. The lesion detection algorithm may include AdaBoost, Deformable Part Models (DPM), Deep Neural Network (DNN), Convolutional Neural Network (CNN), Overfeat, Sparse Coding, and the like. However, it is an exemplary embodiment, and aspects of the present disclosure are not limited thereto.

The classifier 240 may exclude, from the classification, an ultrasonic image from which an ROI is not detected among generated ultrasonic images. For example, suppose that an ROI is detected from four images (images 1 to 4) and not detected from one image (image 5) among five generated ultrasonic images (images 1 to 5). In this case, the classifier 240 classifies the images 1 to 4, except the image 5, and computes a conclusive classification result by combining the classification results for the images 1 to 4.

The display 250 may output a generated ultrasonic image and a conclusive classification result on a screen. The display 250 may output generated ultrasonic images or any ultrasonic image from which an ROI is detected. In addition, the display 250 may output only an ultrasonic image among the ultrasonic images to show features of a lesion.

In addition, the display 250 may output a ROI detection result on the screen. When displaying an ROI detection result, the display 250 may display a detected ROI with a bounding box around the ROI or a cross mark placed at the center thereof, but aspects of the present disclosure are not limited thereto. That is, a detected ROI may be displayed in a manner of using various types of distinguished markers, for example, a circle and a triangle, or coloring the same with various kinds of color.

FIG. 3 is a block diagram illustrating the ultrasonic transmission pattern controller 210 shown in FIG. 2.

Referring to FIG. 3, the ultrasonic transmission pattern 210 includes a pressure sensor 310, a determiner 320, and an energy controller 330.

The pressure sensor 310 may sense pressure applied on the probe 10. According to an exemplary embodiment, the pressure sensor 310 may sense pressure on the probe 10 using a pressure sensor mounted on the probe 10.

Pressure on the probe 10 may be pressure that is applied on the probe 10 from an object's surface in contact with the probe 10, that is, pressure that is made by a user's force to press the probe 10 toward the object. Alternatively, pressure on the probe 10 may be pressure that is applied on the probe 10 from a holder or user thereof, that is, pressure that is made by a user's force to grasp the probe 10.

According to pressure applied on the probe 10, the determiner 320 may determine the number of transmission directions in which ultrasonic signals are to be transmitted.

According to an exemplary embodiment, the determiner 320 may determine the number of transmission directions to be inversely proportional to pressure of the probe 10. For example, suppose that the probe 10 is able to transmit ultrasonic waves in five directions toward an object simultaneously or sequentially. In this case, the determiner 320 classifies a range of pressured to be applied on the probe 10 into three levels (level 1, 2, and 3, and a higher level corresponds to greater pressure). If pressure applied on the probe 10 is level 1, the determiner 320 may determine the number of transmission directions to be 5; if pressure applied on the probe 10 is level 2, the determiner 320 may determine the number of transmission directions to be 3; and if pressure applied on the probe 10 is 3, the determiner 320 may determine the number of transmission directions to be 1.

Based on the determined number of transmission directions, the determiner 320 may determine the transmission directions in which the ultrasonic signals are to be actually transmitted by the probe 10.

According to an exemplary embodiment, from available directions in which the probe 10 is capable of transmitting ultrasonic signals, the determiner 320 may select the transmission directions according to how close an angle is with respect to an axis vertical to an object's surface in contact with the probe 10, wherein the number of the selected transmission directions corresponds to the determined number of transmission directions. For example, in the above-described case, suppose that the probe 10 may transmit ultrasonic waves in five directions (directions 1 to 5) and that an angle with respect to an axis vertical to an object's surface in contact with the probe 10 becomes greater from the direction 1 to the direction 5. If pressure applied on the probe 10 is level 2, the determiner 320 may determine to transmit ultrasonic signals in three directions, and determine transmission directions to be the directions 1, 2, and 3, wherein the directions 1, 2, and 3 are at a closer angle with respect to an axis vertical to the object's surface in contact with the probe 10.

According to another exemplary embodiment, from available directions in which the probe 10 is capable of transmitting ultrasonic signals, the determiner 320 may arbitrarily select the transmission directions, regardless of an angle with respect to an axis vertical to an object's surface in contact with the probe 10.

According to the determined number of transmission directions, the energy controller 330 may control energy of each ultrasonic signal to be transmitted. According to an exemplary embodiment, the energy controller 330 may control energy of each ultrasonic signal to be inversely proportional to the determined number of transmission directions.

When ultrasonic images of an object is obtained using the probe 10, high-definition images may be obtained by increasing energy of each ultrasonic signal to be transmitted to the object. Therefore, when less pressure is applied on the probe 10, the ultrasonic transmission pattern controller 210 reduces energy of each ultrasonic signal to be transmitted and increases the number of transmission directions to obtain a large number of low-definition images. Alternatively, when great pressure is applied on the probe 10, the ultrasonic transmission pattern controller 210 increases energy of each ultrasonic signal to be transmitted and reduces the number of transmission directions to obtain a small number of high-definition images by concentrating ultrasonic energy.

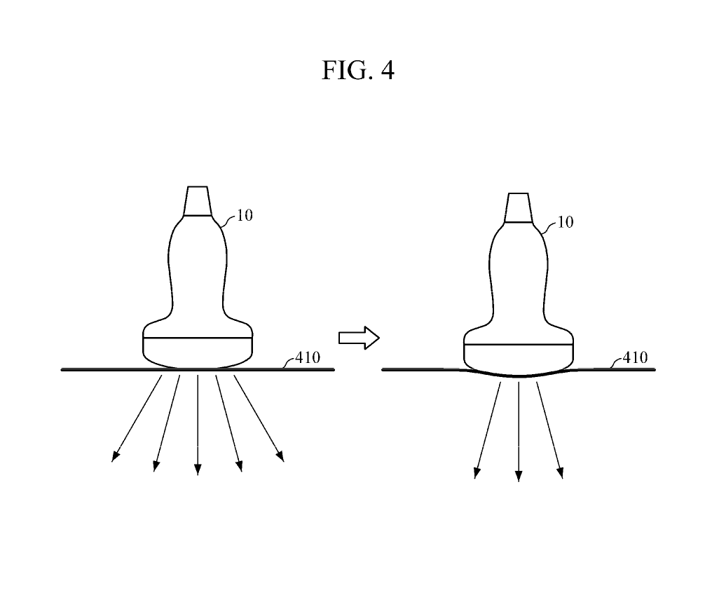

FIG. 4 is a diagram illustrating a number of transmission directions being changed according to a change in pressure applied on the probe 10, according to an exemplary embodiment. It is assumed that the pressure applied on the probe 10 is managed on a level-unit basis and that level 1 to level 5 are assigned according to the magnitude of pressure (that is, the higher the level is, the greater pressure is applied).

Referring to FIG. 4, when the probe 10 is in contact with the surface of an object 410 and pressure applied on the probe 10 from the surface of the object 410 corresponds to level 1, the probe 10 transmits ultrasonic signals with less energy in five directions, as shown in the left-side example of FIG. 4.

Then, if the pressure applied on the probe 10 from the surface of the object 410 increases to level 2, the number of transmission directions is reduced from 5 to 3, but energy is concentrated so that ultrasonic signals, whose energy is greater than when the ultrasonic signals are transmitted in five directions, are transmitted in three directions.

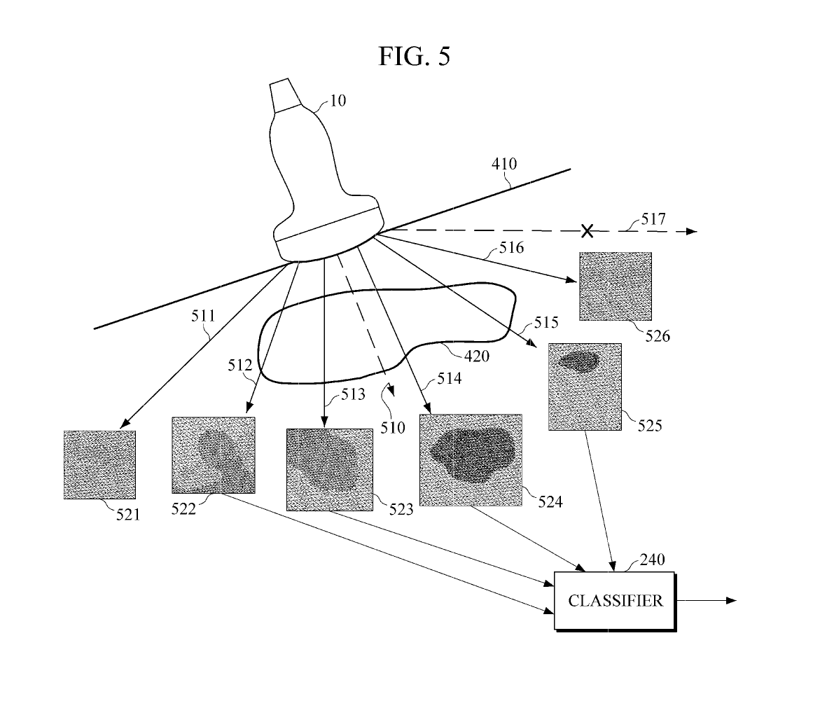

FIG. 5 is a diagram illustrating operations of the CAD apparatus shown in FIG. 2. It is assumed that the probe 10 is capable of transmitting ultrasonic waves in up to seven directions 511 to 517.

Referring to FIGS. 2 and 5, when the probe 10 is in contact with the object 410, the ultrasonic transmission pattern controller 210 may sense pressure applied on the probe 10 and determine the number of transmission directions in which ultrasonic signals are to be transmitted, the transmission directions in which ultrasonic signals are to be transmitted, and energy of each ultrasonic signal according to the sensed pressure. In FIGS. 2 and 5, the ultrasonic transmission pattern controller 210 determines the number of transmission directions to be 6 according to pressure applied on the probe 10, and determines, among seven directions (511 to 517) in which the probe 10 is capable of transmitting ultrasonic signals, six transmission directions (511 to 516), each of which are at a closer angle with respect to an axis 510 vertical to the object's surface in contact with the probe 10.

The probe 10 transmits ultrasonic signals in the six transmission directions 511 to 516 and receives ultrasonic echo signals reflected from the object 410.

Using the ultrasonic echo signals, the image generator 220 generates six ultrasonic images 521 to 526 that correspond to the ultrasonic signals that have been transmitted in the six transmission directions 511 to 516.

The ROI detector 230 may detect an ROI including a lesion 420 from each of the six generated ultrasonic images 521 to 526. In FIG. 5, among the six ultrasonic images 521 to 526, only four ultrasonic images 522 to 525 include the lesion 420, so that an ROI is detected from each of the four ultrasonic images 522 to 525.

The classifier 240 computes a conclusive classification result by performing multi-view classification on the four ultrasonic images 522 to 525 including the lesion 420 among the six ultrasonic images 521 to 526.

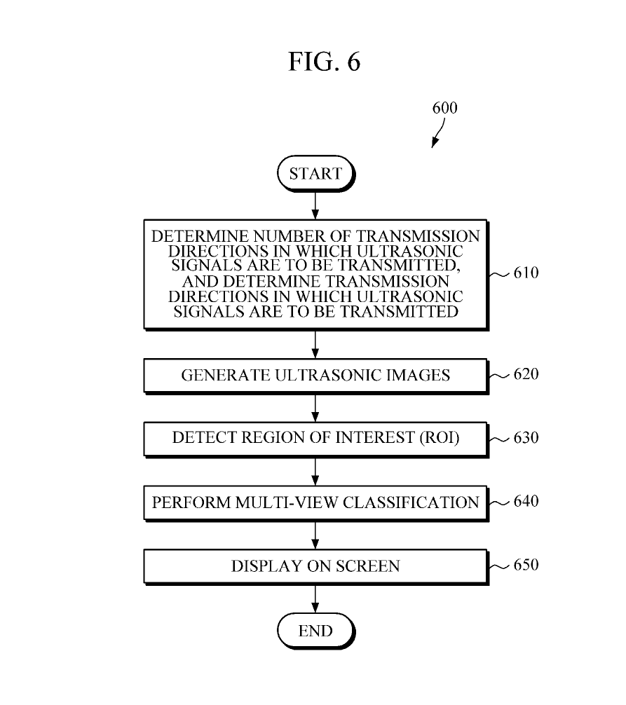

FIG. 6 is a flowchart illustrating a CAD method 600, according to an exemplary embodiment.

Referring to FIG. 6, in operation 610, the CAD method 600 according to an exemplary embodiment includes determining, according to pressure applied on a probe, the number of transmission directions in which ultrasonic signals are to be transmitted and the transmission directions in which ultrasonic signals are to be transmitted. The pressure applied on the probe may be a pressure that is applied on a probe from an object's surface in contact with a probe, that is, pressure that is made by a user's force to press the probe toward the object. Alternatively, pressure on the probe may be pressure that is applied on the probe from a holder thereof, that is, pressure that is made by a user's force to grasp the probe.

In operation 620, based on ultrasonic echo signals that are received in response to the ultrasonic signals transmitted in the determined transmission directions, the CAD method 600 includes generating ultrasonic images of an object. The number of ultrasonic images to be generated may be the same as the determined number of transmission directions in which ultrasonic signals are to be transmitted. For example, when ultrasonic echo signals are received from three directions as the probe 10 transmits ultrasonic signals in the three directions simultaneously or sequentially, the CAD apparatus 20 may generate three ultrasonic images for the three directions, respectively.

In operation 640, the CAD method 600 includes performing multi-view classification on the generated ultrasonic images. For example, the CAD apparatus 20 may extract features from each generated ultrasonic image, perform classification on each ultrasonic image by comparing the extracted features with a pre-stored diagnostic image, and compute a conclusive classification result by combining classification results (e.g., malignancy/benignancy and confidence on determining as malignant/benign) for the generated ultrasonic images.

According to another exemplary embodiment, the CAD method 600 may further include detecting an ROI in operation 630 and displaying on a screen in operation 650.

In operation 630, the CAD method 600 may include detecting an ROI from at least one of the generated ultrasonic images. For example, the CAD apparatus 20 may detect an ROI from each ultrasonic image using a lesion detection algorithm. The ROI may include not only a malignant lesion area, but also a lesion area that is not definitely determined to be malignant/benign or has unusual features. The lesion detection algorithm may include AdaBoost, Deformable Part Models (DPM), Deep Neural Network (DNN), Convolutional Neural Network (CNN), Overfeat, Sparse Coding, and the like. However, the above is an exemplary embodiment, and aspects of the present disclosure are not limited thereto.

In operation 640, any ultrasonic image in which an ROI is not detected in operation 630 among the generated ultrasonic images may be excluded from the classification. For example, suppose that among five ultrasonic images (images 1 to 5), an ROI is detected from four images (images 1 to 4), except one image (image 5). In this case, the CAD apparatus 20 may performs classification only on the images 1 to 4, except the image 5, and compute a conclusive classification result by combining the classification results for the images 1 to 4.

In operation 650, the CAD method 600 may include displaying a generated ultrasonic image and the conclusive classification result on the screen. According to an exemplary embodiment, the CAD apparatus 20 may output the generated ultrasonic images or may output any ultrasonic image from which an ROI is detected. In addition, the CAD apparatus 20 may output only an ultrasonic image among the ultrasonic images to show features of a lesion.

In operation 650, the CAD apparatus 20 may output a detection result of an ROI. According to an exemplary embodiment, the CAD apparatus 20 may display a detected ROI with a bounding box around the ROI or a cross mark placed at the center thereof. However, aspects of the present disclosure are not limited thereto, and the CAD apparatus 20 may display a detected ROI in a manner of using various types of distinguished markers, i.e., a circle and a triangle, or coloring the same with various kinds of color.

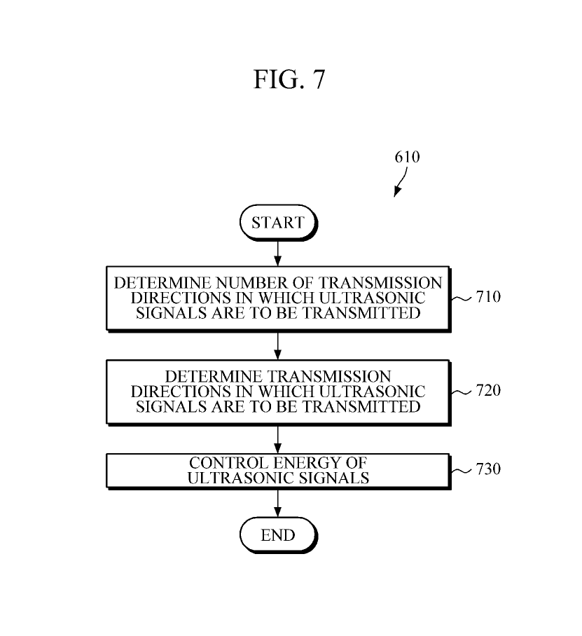

FIG. 7 is a flowchart illustrating the operation 610 of determining a number of transmission directions in which ultrasonic signals are to be transmitted and the transmission directions in which ultrasonic signals are to be transmitted, which is shown in FIG. 6.

Referring to FIG. 7, in operation 710, the operation 610 includes determining the number of transmission directions according to pressure applied on a probe. For example, the CAD apparatus 20 may determine the number of transmission directions to be inversely proportional to pressure on the probe.

In operation 720, based on the determined number of transmission directions, the operation 610 includes determining the transmission directions in which ultrasonic signals are to be transmitted. For example, from available transmission directions in which a probe is capable of transmitting ultrasonic signals, the CAD apparatus 20 may select the transmission directions according to how close an angle is with respect to an axis vertical to an object's surface in contact with the probe, wherein the number of the selected transmission directions corresponds to the determined number of transmission directions. In another example, from available directions from which ultrasonic signals is capable of being transmitted from a probe, the CAD apparatus 20 may arbitrarily select the transmission directions regardless of an angle with respect to an axis vertical to an object's surface in contact with the probe.

In operation 730, according to the determined number of transmission directions, the operation 610 includes controlling energy of the ultrasonic signals to be transmitted from the probe to the object. For example, the CAD apparatus 20 may control energy of each ultrasonic signal to be inversely proportional to the determined number of the transmission directions.

In addition, the exemplary embodiments may also be implemented through computer-readable code and/or instructions on a medium, e.g., a computer-readable medium, to control at least one processing element to implement any above-described embodiments. The medium may correspond to any medium or media which may serve as a storage and/or perform transmission of the computer-readable code.

The computer-readable code may be recorded and/or transferred on a medium in a variety of ways, and examples of the medium include recording media, such as magnetic storage media (e.g., ROM, floppy disks, hard disks, etc.) and optical recording media (e.g., compact disc read only memories (CD-ROMs) or digital versatile discs (DVDs)), and transmission media such as Internet transmission media. Thus, the medium may have a structure suitable for storing or carrying a signal or information, such as a device carrying a bitstream according to one or more exemplary embodiments. The medium may also be on a distributed network, so that the computer-readable code is stored and/or transferred on the medium and executed in a distributed fashion. Furthermore, the processing element may include a processor or a computer processor, and the processing element may be distributed and/or included in a single device.

The foregoing exemplary embodiments are examples and are not to be construed as limiting. The present teaching can be readily applied to other types of apparatuses. Also, the description of the exemplary embodiments is intended to be illustrative, and not to limit the scope of the claims, and many alternatives, modifications, and variations will be apparent to those skilled in the art.

What is claimed is:

1. An apparatus for controlling an ultrasonic transmission pattern of a probe configured to transmit ultrasonic signals to an object in directions, and receive ultrasonic echo signals in response to the ultrasonic signals being reflected from the object, the apparatus comprising:
a determiner configured to:
determine, based on pressure that is applied on the probe, a number of transmission directions in which the ultrasonic signals are to be transmitted; and
determine the transmission directions in which the ultrasonic signals are to be transmitted, based on the determined number of the transmission directions; and
an energy controller configured to control energy of each of the ultrasonic signals based on the determined number of the transmission directions.

2. The apparatus of claim 1, wherein the determiner is further configured to determine the number of the transmission directions to be inversely proportional to the pressure applied on the probe.

3. The apparatus of claim 1, wherein the determiner is further configured to select the transmission directions from available directions in which the probe is capable of transmitting the ultrasonic signals, based on a closeness of an angle between each of the available directions and an axis vertical to a surface of the object in contact with the probe, and
a number of the selected transmission directions corresponds to the determined number of the transmission directions.

4. The apparatus of claim 1, wherein the energy controller is further configured to control the energy of each of the ultrasonic signals to be inversely proportional to the determined number of the transmission directions.

5. The apparatus of claim 1, wherein the pressure applied on the probe is from a surface of the object in contact with the probe, or from a holder of the probe.

6. The apparatus of claim 1, further comprising:
a pressure sensor configured to sense the pressure applied on the probe.

7. A Computer Aided Diagnosis (CAD) apparatus using a probe configured to transmit ultrasonic signals to an object in directions, and receive ultrasonic echo signals in response to the ultrasonic signals being reflected from the object, the apparatus comprising:
an ultrasonic transmission pattern controller configured to:
determine, based on pressure that is applied on the probe, a number of transmission directions in which the ultrasonic signals are to be transmitted; and
determine the transmission directions in which the ultrasonic signals are to be transmitted, based on the determined number of the transmission directions;
an image generator configured to generate ultrasonic images based on the ultrasonic echo signals that are received in response to the ultrasonic signals being transmitted to the object in the determined transmission directions, a number of the generated ultrasonic images corresponding to the determined number of the transmission directions; and
a classifier configured to:
classify each of the generated ultrasonic images; and
combine results of the classification to generate a final result of the classification.

8. The CAD apparatus of claim 7, wherein the ultrasonic transmission pattern controller is further configured to determine the number of the transmission directions to be inversely proportional to the pressure applied on the probe.

9. The apparatus of claim 7, wherein the ultrasonic transmission pattern controller is further configured to select the transmission directions from available directions in which the probe is capable of transmitting the ultrasonic signals, based on a closeness of an angle between each of the available directions and an axis vertical to a surface of the object in contact with the probe, and
a number of the selected transmission directions corresponds to the determined number of the transmission directions.

10. The CAD apparatus of claim 7, wherein the ultrasonic transmission pattern controller is further configured to control energy of each of the ultrasonic signals based on the determined number of the transmission directions.

11. The CAD apparatus of claim 10, wherein the ultrasonic transmission pattern controller is further configured to control the energy of each of the ultrasonic signals to be inversely proportional to the determined number of the transmission directions.

12. The CAD apparatus of claim 7, wherein the pressure applied on the probe is from a surface of the object in contact with the probe, or from a holder of the probe.

13. The CAD apparatus of claim 7, further comprising:
a region of interest (ROI) detector configured to detect an ROI from each of the generated ultrasonic images,
wherein the classifier is further configured to exclude, from the classification, an ultrasonic image in which an ROI is not detected among the generated ultrasonic images.

14. A Computer Aided Diagnosis (CAD) method using a probe configured to transmit ultrasonic signals to an object in directions, and receive ultrasonic echo signals in response to the ultrasonic signals being reflected from the object, the method comprising:
determining, based on pressure that is applied on the probe, a number of transmission directions in which the ultrasonic signals are to be transmitted;
determining the transmission directions in which the ultrasonic signals are to be transmitted, based on the determined number of the transmission directions;
generating ultrasonic images based on the ultrasonic echo signals that are received in response to the ultrasonic signals being transmitted to the object in the determined transmission directions, a number of the generated ultrasonic images corresponding to the determined number of the transmission directions;
classifying each of the generated ultrasonic images; and
combining results of the classification to generate a final result of the classification.

15. The CAD method of claim 14, wherein the determining the number of the transmission directions comprises determining the number of the transmission directions to be inversely proportional to the pressure applied on the probe.

16. The CAD method of claim 14, wherein the determining the transmission directions comprises selecting the transmission directions from available directions in which the probe is capable of transmitting the ultrasonic signals, based on a closeness of an angle between each of the available directions and an axis vertical to a surface of the object in contact with the probe, and
a number of the selected transmission directions corresponds to the determined number of the transmission directions.

17. The CAD method of claim 14, further comprising controlling energy of each of the ultrasonic signals based on the determined number of the transmission directions.

18. The CAD method of claim 17, wherein the controlling of energy comprises controlling the energy of each of the ultrasonic signals to be inversely proportional to the determined number of the transmission directions.

19. The CAD method of claim 14, wherein the pressure applied on the probe is from a surface of the object in contact with the probe, or from a holder of the probe.

20. The CAD method of claim 14, further comprising:
detecting a region of interest (ROI) from each of the generated ultrasonic images,
wherein the classifying comprises excluding, from the classification, an ultrasonic area in which an ROI is not detected among the generated ultrasonic images.
